(19) United States
(12) Patent Application Publication
(10) Pub. No.: US 2025/0288348 A1
(43) Pub. Date: Sep. 18, 2025

(54) ANTI-BACKDRIVE MECHANISM FOR VESSEL SEALING INSTRUMENT

(71) Applicant: Covidien LP, Mansfield, MA (US)

(72) Inventors: Grant T. Sims, Boulder, CO (US); William R. Whitney, Boulder, CO (US); Jason G. Weihe, Longmont, CO (US); James D. Allen, IV, Broomfield, CO (US); Jason T. Sanders, Longmont, CO (US); Alyssa M. Sawyer, Broomfield, CO (US); Curtis M. Siebenaller, Frederick, CO (US)

(73) Assignee: Covidien LP, Mansfield, MA (US)

(21) Appl. No.: 19/222,213

(22) Filed: May 29, 2025

Related U.S. Application Data
(62) Division of application No. 17/849,048, filed on Jun. 24, 2022.
(60) Provisional application No. 63/214,939, filed on Jun. 25, 2021.

(51) Int. Cl. A61B 18/14; A61B 17/29; A61B 18/00; A61B 18/12

(52) U.S. Cl. CPC A61B 18/1447; A61B 18/1206; A61B 2017/2936; A61B 2018/00208; A61B 2018/0063; A61B 2018/1455

(57) ABSTRACT

A vessel sealing instrument includes a housing having a shaft extending from a distal end thereof, the distal end including an end effector assembly having a pair of opposing jaw members operably coupled thereto. One or both of the jaw members is moveable between open and closed positions for clamping tissue with a closure pressure. One or both of the jaw members connects to a generator that provides energy thereto in accordance with a sealing algorithm upon activation thereof. An anti-backdrive mechanism is coupled to the end effector assembly and includes a drive shaft coupled at one end to a solenoid and another end that engages one of the jaw members upon extension thereof to provide additional closure pressure between the jaw members. The drive shaft is extendible by the solenoid to extend the drive shaft in response to tissue expansion during sealing based on the sealing algorithm.

### CROSS REFERENCE TO RELATED APPLICATION

This application is a divisional of U.S. patent application Ser. No. 17/849,048, filed Jun. 24, 2022, which claims the benefit of U.S. Provisional Application Ser. No. 63/214,939 filed Jun. 25, 2021, the entire contents of which being incorporated by reference herein. To the extent appropriate a claim of priority is made to each of the above-disclosed applications.

### FIELD

The present disclosure relates to surgical instruments and, more particularly, to anti-backdrive mechanisms for vessel sealing instruments configured to maintain closure pressure during sealing.

### BACKGROUND

A surgical forceps is a pliers-like surgical instrument that relies on mechanical action between its jaw members to grasp, clamp, and constrict tissue. Electrosurgical forceps utilize both mechanical clamping action and energy to heat tissue to treat, e.g., coagulate, cauterize, or seal, tissue. Typically, once tissue is grasped under a closure pressure suitable to seal vessels or tissue, the actuation mechanism (e.g., handle) is locked during the delivery of electrosurgical energy to produce a seal. In some instance the surgeon holds the actuation mechanism during electrosurgical activation. During sealing, the tissue naturally expands against the closure pressure which, in some instances, can affect the resulting tissue seal as the closure pressure no longer falls within a particular closure pressure range.

Accordingly, there exists a need to maintain the closure pressure within the desired closure pressure range during the sealing process.

### SUMMARY

As used herein, the term “distal” refers to the portion that is being described which is further from a user, while the term “proximal” refers to the portion that is being described which is closer to a user. Further, to the extent consistent, any or all of the aspects detailed herein may be used in conjunction with any or all of the other aspects detailed herein.

Provided in accordance with aspects of the present disclosure is a vessel sealing instrument including a housing having a shaft extending from a distal end thereof. A distal end of the shaft includes an end effector assembly having a pair of opposing first and second jaw members operably coupled thereto. One or both of the first or second jaw members is moveable between an open position and a closed position for clamping tissue with a closure pressure within the range of about 3 kg/cm² to about 16 kg/cm². One or both of the first or second jaw members is adapted to connect to a generator configured to provide electrosurgical energy thereto in accordance with a sealing algorithm upon activation thereof.

An anti-backdrive mechanism is operably associated with the end effector assembly and includes a drive shaft operably coupled at a proximal end to a solenoid and including a distal end configured to operably engage one or both of the first and second jaw members upon extension thereof to provide additional closure pressure between the jaw members. The drive shaft is selectively extendible by the solenoid to extend the drive shaft in response to tissue expansion during sealing based on the sealing algorithm.

In aspects according to the present disclosure, upon extension of the drive shaft in a first direction, the drive shaft engages an abutting surface disposed on a proximal end of the first jaw member. In other aspects according to the present disclosure, the first jaw member is rotatable relative to the second jaw member about a pivot and wherein the abutting surface is offset relative to the pivot. In yet other aspects according to the present disclosure, upon extension of the drive shaft in a second direction, the drive shaft retracts relative to the first jaw member allowing the first jaw member to open relative to the second jaw member.

In aspects according to the present disclosure, the solenoid extends the drive shaft in response to tissue expansion during sealing based on the sealing algorithm, the drive shaft configured to maintain the closure pressure between jaw members within the range of about 3 kg/cm² to about 16 kg/cm².

Provided in accordance with aspects of the present disclosure is a vessel sealing instrument including a housing having a shaft extending from a distal end thereof. A distal end of the shaft includes an end effector assembly having a pair of opposing first and second jaw members operably coupled thereto pivotable about a pivot between an open position and a closed position for clamping tissue with a closure pressure within the range of about 3 kg/cm² to about 16 kg/cm².

An anti-backdrive mechanism is operably associated with the end effector assembly, the anti-backdrive mechanism including a pivot block operably supporting the pivot and translatable within the first jaw member. A drive shaft is operably coupled at a proximal end to a controller and at a distal end to the pivot block. The controller is configured to extend the drive shaft to move the pivot block distally to adjust a moment about the pivot and provide additional closure pressure between the first and second jaw members.

In aspects according to the present disclosure, the controller is configured to continually monitor the closure pressure between the first and second jaw members and adjust the drive shaft in accordance thereto. In other aspects according to the present disclosure, the controller is configured to extend the drive shaft in response to the closure pressure falling outside the range of about 3 kg/cm² to about 16 kg/cm².

In aspects according to the present disclosure, the pivot block is only moveable when the first and second jaw members are disposed in the closed position.

Provided in accordance with aspects of the present disclosure is a vessel sealing instrument including a housing having a shaft extending from a distal end thereof. A distal end of the shaft includes an end effector assembly having a pair of opposing first and second jaw members operably coupled thereto. One or both of the first or second jaw members is pivotable about a pivot between an open position and a closed position for clamping tissue with a closure pressure within the range of about 3 kg/cm² to about 16 kg/cm².

An anti-backdrive mechanism is operably associated with the end effector assembly and includes a pivot block operably supporting the pivot and translatable within a cavity defined within the first jaw member. A pre-loaded spring is anchored at a proximal end to the cavity and at a distal end to the pivot block. The pre-loaded spring is configured to move the pivot block to adjust a moment about the pivot and provide additional closure pressure between the first and second jaw members.

In aspects according to the present disclosure, the pivot block is only moveable by the pre-loaded spring when the first and second jaw members are disposed in the closed position.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1A is a perspective view of an electrosurgical forceps provided in accordance with the present disclosure having in-line electrosurgical activation;

FIG. 1B is a perspective view of an electrosurgical forceps provided in accordance with another embodiment of the present disclosure having a ratchet-like handle assembly;

FIG. 2A is an enlarged, perspective view of an end effector assembly of the electrosurgical forceps of FIG. 1 wherein first and second jaw members of the end effector assembly are disposed in a spaced-apart position;

FIG. 2B is an enlarged, perspective view of the end effector assembly of FIG. 2A wherein the first and second jaw members are disposed in an approximated position;

FIG. 3A is a side view of a proximal portion of the electrosurgical forceps of FIG. 1 with a movable handle and trigger thereof disposed in respective un-actuated positions;

FIG. 3B is a side view of the proximal portion of the electrosurgical forceps shown in FIG. 3A with the movable handle disposed in an actuated position and the trigger disposed in the un-actuated position;

FIG. 3C is a side view of the proximal portion of the electrosurgical forceps shown in FIG. 3A with the movable handle and trigger disposed in respective actuated positions;

FIG. 4A is a side view of another proximal portion of the electrosurgical forceps of FIG. 1 with portions removed to illustrate a trigger assembly thereof with the trigger disposed in the un-actuated position;

FIG. 4B is a side view of the proximal portion of the electrosurgical forceps shown in FIG. 4A with portions removed to illustrate the trigger assembly with the trigger disposed in the actuated position;

FIGS. 5A and 5B is an enlarged, schematic side view of an end effector assembly having a solenoid anti-backdrive mechanism provided in accordance with one embodiment of the present disclosure;

FIGS. 6A-6B are enlarged, schematic side views of an end effector assembly having two respective embodiments of a pivot block anti-backdrive mechanism provided in accordance with the present disclosure; and

FIG. 7 is an enlarged, schematic side view of an end effector assembly having a toggle-like anti-backdrive mechanism provided in accordance with one embodiment of the present disclosure.

### DETAILED DESCRIPTION

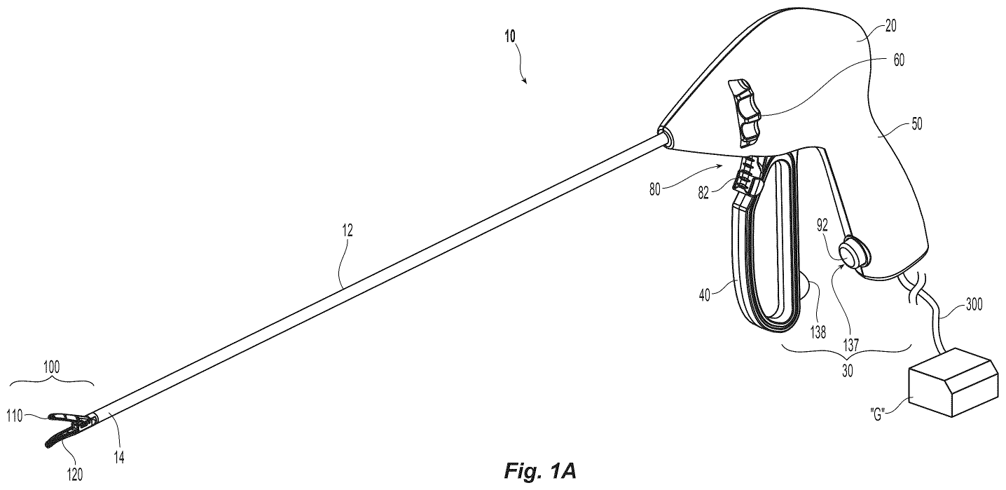

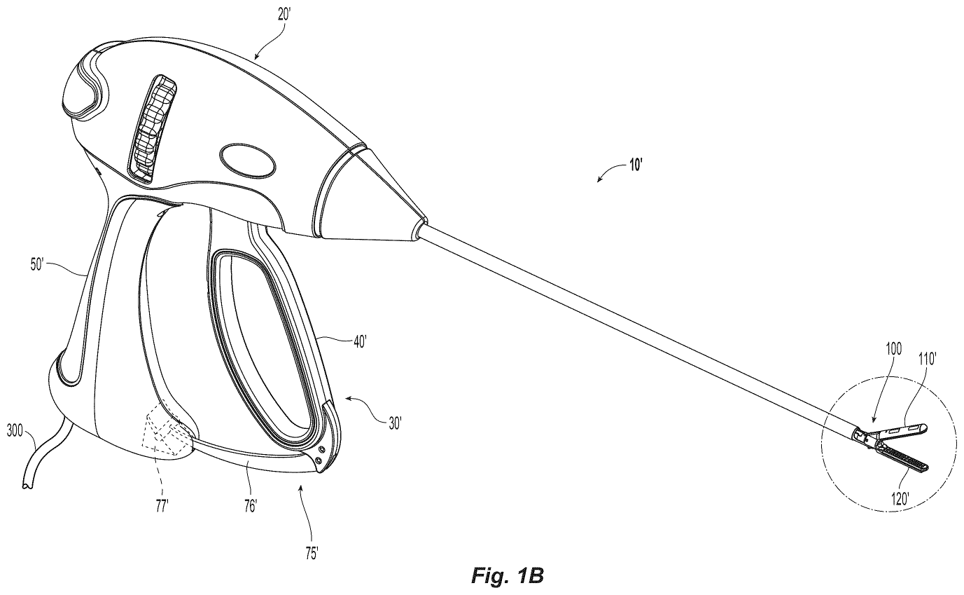

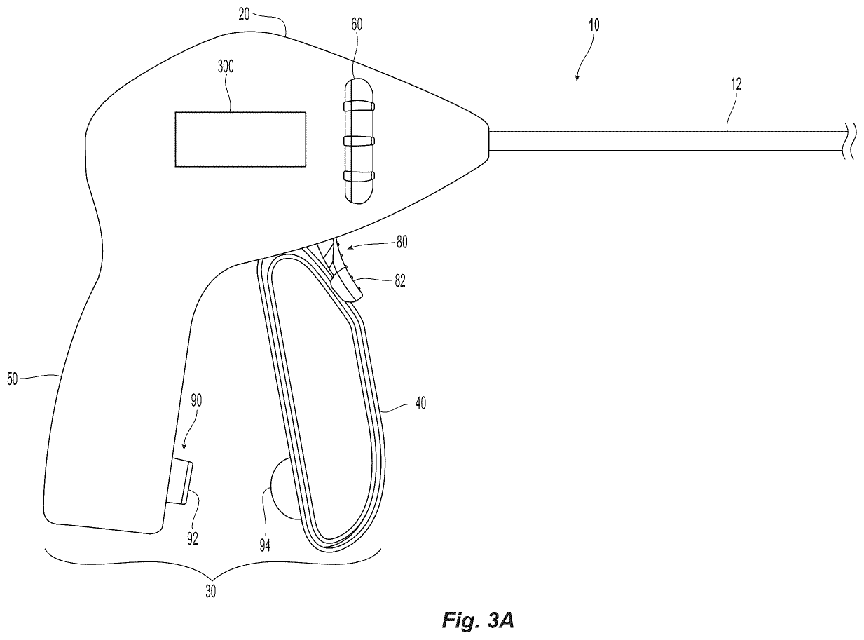

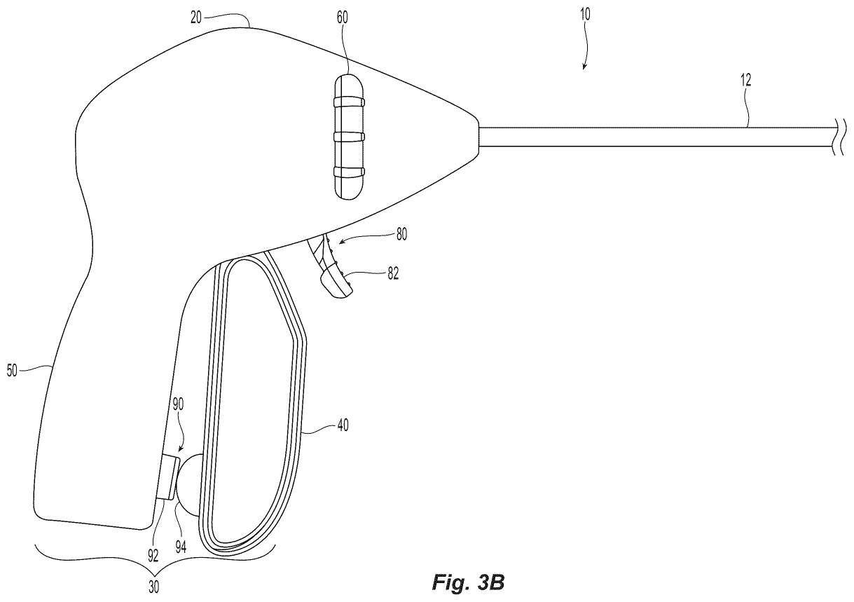

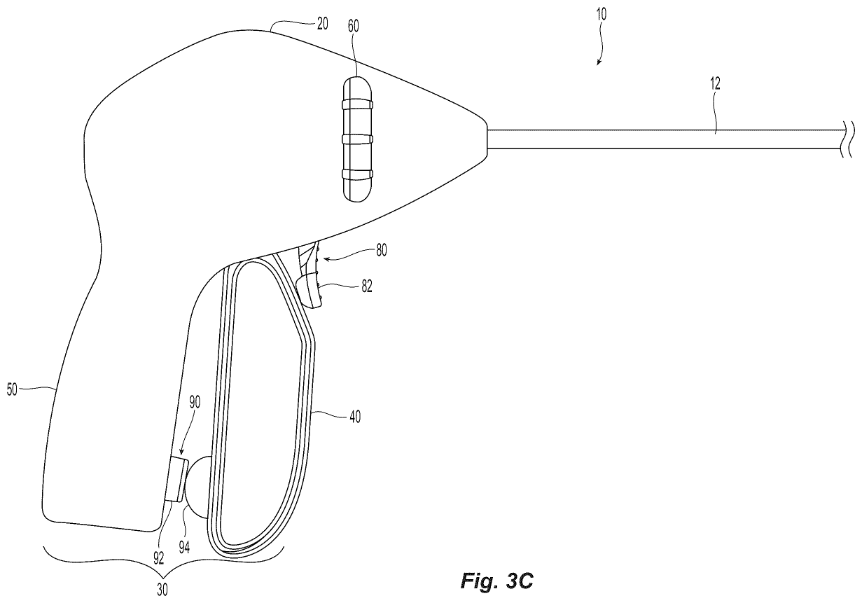

Referring to FIG. 1A, a surgical instrument provided in accordance with the present disclosure is shown configured as a bipolar electrosurgical forceps 10 for use in connection with endoscopic surgical procedures, although the present disclosure may be equally applicable for use with other surgical instruments such as those for use in endoscopic and/or traditional open surgical procedures. Forceps 10 generally includes a housing 20, a handle assembly 30, a rotating assembly 60, a trigger assembly 80, an activation assembly 90 (FIGS. 3A-3C), and an end effector assembly 100 including first and second jaw members 110, 120.

Forceps 10 further includes a shaft 12 having a distal end portion 14 configured to engage (directly or indirectly) end effector assembly 100 and a proximal end portion 16 that engages (directly or indirectly) housing 20. Rotating assembly 60 is rotatable in either direction to rotate shaft 12 and end effector assembly 100 relative to housing 20 in either direction. Housing 20 houses the internal working components of forceps 10.

An electrosurgical cable 300 connects forceps 10 to an electrosurgical generator “G” or other suitable energy source, although forceps 10 may alternatively be configured as a handheld instrument incorporating energy-generating and/or power components thereon or therein. Cable 300 includes wires (not shown) extending therethrough, into housing 20, and through shaft 12, to ultimately connect electrosurgical generator “G” to jaw member 110 and/or jaw member 120 of end effector assembly 100. Activation button 92 of activation assembly 90 is disposed on housing 20 are electrically coupled between end effector assembly 100 and cable 300 to enable the selective supply of energy to jaw member 110 and/or jaw member 120, e.g., upon activation of activation button 92. However, other suitable electrical connections and/or configurations for supplying electrosurgical energy to jaw member 110 and/or jaw member 120 may alternatively be provided, as may other suitable forms of energy, e.g., ultrasonic energy, microwave energy, light energy, thermal energy, etc.

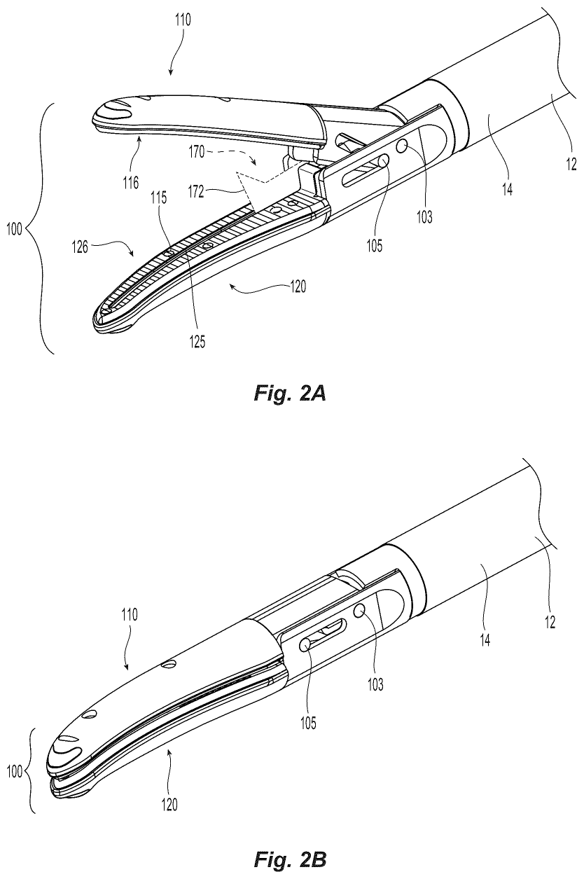

Forceps 10 additionally includes a knife assembly 170 (FIG. 2A) operably coupled to trigger assembly 80 and extending through housing 20 and shaft 12. One or both of jaw members 110, 120 defines a knife channel 125 (FIG. 2A) configured to permit reciprocation of a knife blade 172 (FIG. 2A) of knife assembly 170 (FIG. 2A) therethrough, e.g., in response to actuation of trigger 82 of trigger assembly 80. Trigger assembly 80 is described in greater detail below as are other embodiments of trigger assemblies configured for use with forceps 10.

With additional reference to FIGS. 2A and 2B, end effector assembly 100, as noted above, is disposed at distal end portion 14 of shaft 12 and includes a pair of jaw members 110 and 120 pivotable between a spaced-apart position and an approximated position for grasping tissue therebetween. End effector assembly 100 is designed as a unilateral assembly, e.g., wherein one of the jaw members 120 is fixed relative to shaft 12 and the other jaw member 110 is movable relative to both shaft 12 and the fixed jaw member 120. However, end effector assembly 100 may alternatively be configured as a bilateral assembly, e.g., wherein both jaw member 110 and jaw member 120 are movable relative to one another and with respect to shaft 12.

Each jaw member 110, 120 of end effector assembly 100 includes an electrically-conductive tissue-contacting surface 116, 126. Tissue-contacting surfaces 116 are positioned to oppose one another for grasping and treating tissue. More specifically, tissue-contacting surfaces 116, 126 are electrically coupled to the generator “G,” e.g., via cable 300, and activation button 92 to enable the selective supply of energy thereto for conduction through tissue grasped therebetween, e.g., upon activation of activation button 92. One or both of tissue-contacting surfaces 116, 126 may include one or more stop members 115 extending therefrom to define a minimum gap distance between electrically-conductive tissue-contacting surfaces 116, 126 in the approximated position of jaw members 110, 120, facilitate grasping of tissue, and/or inhibit shorting between electrically-conductive tissue-contacting surfaces 116, 126.

The stop member(s) 115 may be formed at least partially from an electrically-insulative material or may be effectively insulative by electrically isolating the stop member(s) from one or both of the electrically-conductive tissue-contacting surfaces 116, 126. The one or more stop members 115 may be disposed on one or both jaw members 110, 120 or on the tissue-contacting surfaces 116, 126 and are configured to regulate the distance therebetween. Details relating to various stop member designs are disclosed in U.S. Pat. Nos. 7,857,812, 10,687,887 the entire contents of each of which being incorporated by reference here.

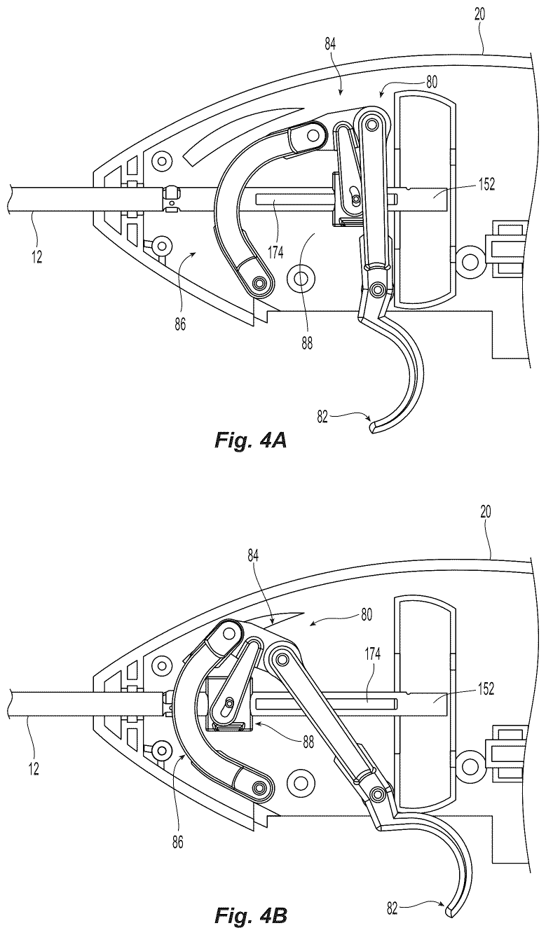

A pivot pin 103 of end effector assembly 100 extends transversely through aligned apertures defined within jaw members 110, 120 and shaft 12 to pivotably couple jaw member 110 to jaw member 120 and shaft 12. A cam pin 105 of end effector assembly 100 extends transversely through cam slots defined within jaw members 110, 120 and is operably engaged with a distal end portion of a drive bar 152 (FIGS. 4A and 4B) of a drive assembly 300 (only drive bar 152 (FIGS. 4A and 4B) of the drive assembly 300 is shown and drive assembly is generically represent by component 300 in FIG. 3A) such that longitudinal translation of drive bar 152 (FIGS. 4A and 4B) through shaft 12 translates cam pin 105 relative to jaw members 110, 120. Various drive assemblies are shown and described with respect to commonly-owned U.S. Pat. Nos. 7,857,812, 8,540,711, 7,384,420, 7,090,673, 7,101,372, 7,255,697, 7,101,371, 7,131,971, 7,083,618, and 10,842,553, the entire contents of each of which being incorporated by reference herein.

More specifically, distal translation of cam pin 105 relative to jaw members 110, 120 urges cam pin 105 distally through the cam slots to thereby pivot jaw members 110, 120 from the spaced-apart position towards the approximated position, although cam slots may alternatively be configured such that proximal translation of cam pin 105 pivots jaw members 110, 120 from the spaced-apart position towards the approximated position. One suitable drive assembly is described in greater detail, for example, in U.S. Pat. No. 9,655,673, the entire contents of which are hereby incorporated herein by reference.

Referring to FIGS. 1A-3C, handle assembly 30 includes a fixed handle 50 and an actuator, e.g., movable handle 40. Fixed handle 50 is integrally associated with housing 20 and movable handle 40 is movable relative to fixed handle 50. Movable handle 40 is ultimately connected to the drive assembly (not shown) that, together, mechanically cooperate to impart movement of jaw members 110 and 120 between the spaced-apart and approximated positions to grasp tissue between electrically-conductive surfaces 116, 126, respectively. More specifically, pivoting of movable handle 40 relative to fixed handle 50 from an un-actuated position towards an actuated position pivots jaw members 110, 120 from the spaced-apart position towards the approximated position. On the other hand, when movable handle 40 is released or returned towards the initial position relative to fixed handle 50, jaw members 110, 120 are returned towards the spaced-apart position.

A biasing spring (not shown) associated with movable handle 40 and/or the drive assembly may be provided to bias jaw members 110, 120 towards a desired position, e.g., the spaced-apart position or the approximated position. Various drive assemblies are shown and described in any one of the above-identified commonly-owned U.S. Patents referenced herein.

Fixed handle 50 operably supports activation button 92 of activation assembly 90 thereon in an in-line position, wherein activation button 92 is disposed in the actuation path of movable handle 40. In this manner, upon pivoting of movable handle 40 relative to fixed handle 50 from the actuated position to an activated position, protrusion 94 of movable handle 40 is urged into contact with activation button 92 to thereby activate activation button 92 and initiate the supply of energy to electrically-conductive surfaces 116, 126, e.g., to treat tissue grasped therebetween. Alternatively, actuation button 92 may be disposed in any other suitable position, on housing 20 or remote therefrom, to facilitate manual activation by a user to initiate the supply of energy to electrically-conductive surfaces 116, 126.

With reference to FIGS. 1A-2B and 4A-4B, as noted above, trigger assembly 80 is operably coupled to knife blade 172 of knife assembly 170. More specifically, trigger 82 of trigger assembly 80 is selectively actuatable, e.g., from an un-actuated position (FIGS. 3A and 4A) to an actuated position (FIGS. 3C and 4B), to deploy knife blade 172 distally through jaw members 110, 120 to cut tissue grasped between electrically-conductive surfaces 116, 126. Knife assembly 170 includes knife blade 172 and a knife bar 174 engaged with and extending proximally from knife blade 172 through shaft 12 and drive bar 152 into housing 20 where knife bar 174 is operably coupled with trigger assembly 80, as detailed below.

Referring to FIGS. 4A and 4B, trigger assembly 80 includes trigger 82, a link 84, e.g., a T-link 84, a link 86, e.g., an arcuate linkage 86 although other configurations, e.g., linear, angled, etc. are also contemplated, and a slider block 88. In this manner, trigger assembly 80 defines a four-bar mechanical linkage assembly for driving slider block 88 to actuate the knife blade 172. This and other types of trigger mechanism are also contemplated such as, for example, trigger mechanisms described in any one of the above-identified commonly-owned U.S. Patents referenced herein or U.S. patent application Ser. No. 16/558,477, the entire contents of each of which being incorporated by reference herein.

As mentioned above, pivoting of movable handle 40 relative to fixed handle 50 from an un-actuated position towards an actuated position pivots jaw members 110, 120 from the spaced-apart position towards the approximated position for grasping tissue therebetween.

When fully grasped, the drive assembly 300 is configured to initially generate a closure pressure suitable for sealing vessels upon activation of electrosurgical energy from generator “G”. Maintaining closure pressures within the range of about 3 Kg/cm² to about 16 Kg/cm² are known to promote quality seals.

With in-line actuation instruments, the surgeon is typically required to maintain the handle 40 in position to continually maintain the closure pressure. For example and as shown in FIG. 1A, handle 40 is initially actuated under a light pressure to grasp and manipulate tissue prior to sealing as the jaw members 110, 120 may be closed without fully actuating handle 40 relative to handle 50. Once the tissue is properly positioned between jaw members 110, 120, the handle 40 may be fully actuated to close the jaw members 110, 120 about tissue within the above-noted pressure range and simultaneously activate the forceps 10 for sealing. With this type of forceps 10, the surgeon must maintain the handle 40 fully actuated to maintain the initial closure pressure. This is known as in-line activation.

Other forceps e.g., forceps 10′ of FIG. 1B include handle assemblies 30′ (including moveable handle 40′ and fixed handle 50′) that have a ratchet-like locking system 75′ affixed to a portion of the housing 20′ or handle assembly 30′ which is configured to lock handle 40′ relative to the fixed handle 50′ to initially generate and maintain the appropriate closure pressures between jaw members 110′, 120′ when locked. Ratchet-like locking system 75′ includes a flange 76′ extending from handle 40′ configured to mechanically engage and lock within a corresponding ramp 77′ (shown in phantom) disposed within handle 50′. Various such forceps and handle assemblies are shown in any one of the above-identified commonly-owned U.S. Patents referenced herein.

After the initial closure pressure within the above-identified range is generated and the jaw members 110, 120 (or 110′, 120′) are clamped on a vessel or on tissue, the forceps 10 (10′) is ready for activation. As mentioned above, during sealing the vessel or tissue expands against the jaw members, e.g., jaw members 110′, 120′, which may reduce the actual closure pressure during formation of the seal. If the closure pressure falls outside of the above-noted range, the seal may not be as effective.

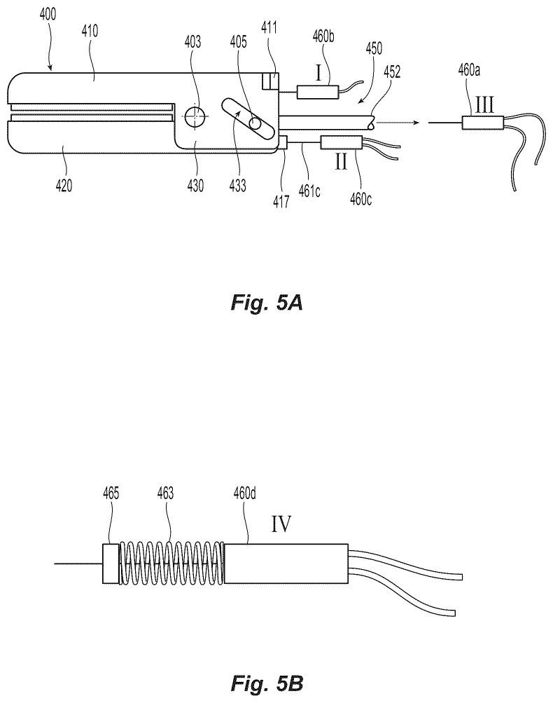

FIGS. 5A-7 show various end effector assemblies which include one or more so-called “anti-backdrive assemblies” configured to maintain the required closure pressure during the sealing process. All of the below-described anti-backdrive assemblies are configured to provide additional pressure to the jaw members, e.g., jaw members 110, 120, to offset the forces attributed to tissue expansion and hold the jaw members 110, 120 in position relative to one another. It is envisioned that any of the below-described anti-backdrive assemblies may be: passive, e.g., prevent the jaw members from 110, 120 from moving during tissue expansion; proactive, e.g., anticipate tissue expansion and counteract the same; or reactive, e.g., measure the expansion or rate of expansion (feedback) and counteract the same.

FIGS. 5A-5B shows one embodiment of an end effector assembly 400 having a jaw member 410 equipped with an anti-backdrive assembly 450. Anti-backdrive assembly 450 includes a solenoid controller individually referred to as solenoid controllers 460a, 460b and 460c that each provide additional closure pressure to the jaw members 410, 420 as needed in any of the fashions described above, e.g., proactively, anticipatorily, or passively. One, two or all three controllers 460a, 460b, 460c may be utilized individually or simultaneously for this purpose.

Jaw member 410 of end effector assembly 400 cooperates with a drive rod 452 actuatable via handle 40 to move the jaw members 410, 420 between open and closed positions upon translation thereof by virtue of a cam pin 405 engaging a cam slot 433 defined in a proximal flange 430 of jaw member 410. When configured for use with solenoid controller 460a, the drive rod 452 is operably coupled to the solenoid controller 460a such that, upon request or in accordance with a sealing algorithm, the solenoid controller 460a provides additional closure force to the jaw members 410, 420 by further translating the drive rod 452 which, in turn, further actuates the cam pin 405 within cam slot 433 thereby increasing closure pressure and holding the jaw members 110, 120 relative to one another.

One or more sensors or other types of feedback mechanisms (not shown) may be utilized to communicate with the solenoid controller 460a to regulate the additional closure pressure to ensure the closure pressure continually falls within the above-identified closure pressure range during the entire sealing process. Alternatively, the solenoid controller 460a may be configured to cooperate with a sealing algorithm and regulated accordingly to apply additional closure pressure in accordance therewith.

Solenoid controller 460a may continually monitor the jaw members 410, 420 for feedback and adjust the drive rod 452 accordingly to maintain the appropriate closure pressure between the jaw members 410, 420 during the entire sealing process. Various types of sensors (not shown) or algorithms may be utilized for this purpose. Once sealed, the drive rod 452 is fully retracted to allow the jaw members 410, 420 to open via handle 40 (or 40′).

Solenoid controller 460b is a variation on solenoid controller 460a and although configured to operate in a slightly different fashion, shares many of the same characteristics with solenoid controller 460a in that solenoid controller 460b may be used with one or more sensors, feedback systems, or cooperate with a sealing algorithm to regulate the additional closure pressure. More particularly, flange 430 of jaw member 410 includes an abutting surface 411 on a proximal-facing side thereof located above-center of the pivot 403 such that, after the jaw members 410, 420 are closed about tissue via actuation of pin 405 within cam slot 433 defined within the flange 430, the solenoid controller 460b extends a distal end 462b of a drive shaft 461b into engagement with abutting surface 411 to further cam jaw member 410 about pivot 403 to increase closure pressure as needed. The solenoid controller 460b may extend the drive shaft 461b upon: user request; based on a sealing algorithm; on reaction to feedback from the jaw members 410, 420 or tissue disposed therebetween; or in anticipation of tissue expansion during sealing.

The controller 460b may continually monitor the jaw members 410, 420 for feedback and adjust the drive shaft 461b accordingly to maintain the appropriate closure pressure between the jaw members 410, 420 during the entire sealing process. Various types of sensors (not shown) or algorithms may be utilized for this purpose. Once sealed, the drive shaft 461b is fully retracted to allow the jaw members 410, 420 to open via handle 40 (or 40′).

Solenoid controller 460c is yet another variation on solenoid controller 460a and although configured to operate in a slightly different fashion, shares many of the same characteristics with solenoid controllers 460a, 460b in that solenoid controller 460c may also be used with one or more sensors, feedback systems, or cooperate with a sealing algorithm to regulate the additional closure pressure. More particularly, flange 430 of jaw member 410 includes a proximally extending rib 417 that is configured to engage a drive shaft 461c of solenoid controller 460c. Rib 417 is disposed on the proximal-facing side of flange 430 located below-center of the pivot 403 such that, after the jaw members 410, 420 are closed about tissue via actuation of pin 405 within cam slot 433 defined within the flange 430, the solenoid controller 460c retracts the drive shaft 461c which pulls rib 417 to further cam jaw member 410 about pivot 403 to increase closure pressure as needed. The solenoid controller 460c may retract the drive shaft 461c upon: user request; based on a sealing algorithm; on reaction to feedback from the jaw members 410, 420 or tissue disposed therebetween; or in anticipation of tissue expansion during sealing.

The controller 460c may continually monitor the jaw members 410, 420 for feedback and adjust the drive shaft 461c accordingly to maintain the appropriate closure pressure between the jaw members 410, 420 during the entire sealing process. Various types of sensors (not shown) or algorithms may be utilized for this purpose. Once sealed, the drive shaft 461c is fully extended to allow the jaw members 410, 420 to open via handle 40 (or 40′) as explained above.

FIG. 5B shows another version of the solenoid 460d having a spring 463 disposed between a distal end of the solenoid 460d and a return block 465. The spring 463 and the solenoid 460d may cooperate to hold the jaw members 110, 120 relative to one another.

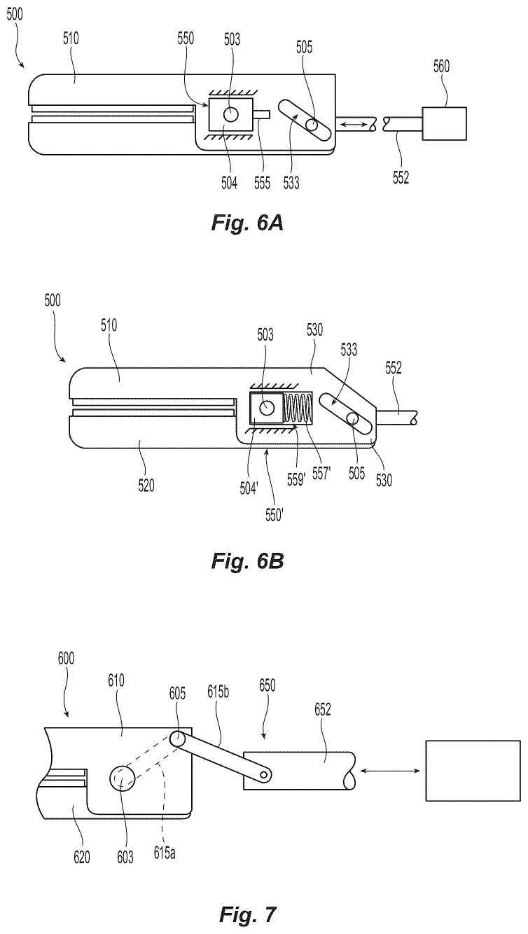

Turning now to FIGS. 6A and 6B which show other embodiments of anti-backdrive mechanisms 550, 550′ for use with forceps 10, 10′. Anti-backdrive mechanisms 550, 550′ are similar to anti-backdrive mechanism 450 and, as such, only those differences will be discussed below. Moreover, end effector 500 is similar with respect to FIGS. 6A and 6B and, as such, only those differences are described with respect to the anti-backdrive mechanism 550′ are discussed in detail.

Jaw member 510 of end effector assembly 500 cooperates with a drive rod 552 actuatable via handle 40 to move the jaw members 510, 520 between open and closed positions upon translation thereof by virtue of a cam pin 505 engaging a cam slot 533 defined in a proximal flange 530 of jaw member 510. Anti-backdrive mechanism 550 includes a pivot pin block 504 that operably supports pivot pin 503 therein. Pivot pin block 504 is configured to translate via drive shaft 555 to provide additional closure pressure between jaw members 510, 520 by adjusting the moment of force or torque associated therewith.

After the jaw members 510, 520 are closed about tissue via actuation of pin 505 within cam slot 533 defined within the flange 530, a controller 560 retracts the drive shaft 555 to move the pivot pin block 504 proximally to further cam jaw member 510 thereabout to increase the moment of force and therefor the closure pressure as needed. The controller 560 may retract the drive shaft 555 upon: user request; based on a sealing algorithm; on reaction to feedback from the jaw members 510, 520 or tissue disposed therebetween; or in anticipation of tissue expansion during sealing.

The controller 560 may continually monitor the jaw members 510, 520 for feedback and adjust the drive shaft 555 accordingly to maintain the appropriate closure pressure between the jaw members 510, 520 during the entire sealing process. Various types of sensors (not shown) or algorithms may be utilized for this purpose. Once sealed, the drive shaft 555 is fully extended to allow the jaw members 510, 520 to open via handle 40 (or 40′).

FIG. 6B shows a similar end effector assembly 500 having a different anti-backdrive mechanism 550′. Jaw member 510 of end effector assembly 500 cooperates with a drive rod 552 actuatable via handle 40 to move the jaw members 510, 520 between open and closed positions upon translation thereof by virtue of a cam pin 505 engaging a cam slot 533 defined in a proximal flange 530 of jaw member 510. Anti-backdrive mechanism 550′ includes a pivot pin block 504′ that operably supports pivot pin 503′ within a cavity 559′ defined in the flange 530 therein. Pivot pin block 504′ is operably engaged to a preloaded spring 557′ configured to provide additional closure pressure by moving the pivot pin 503′ to offset the moment of force or torque associated therewith. More particularly, once the jaw members 510, 520 are closed about tissue under the above-identified closure pressure and the jaw members 510, 520 are energized to seal tissue, any forces associated with tissue expansion are offset by the additional closure pressure associated with the spring 557′.

Anti-backdrive mechanism 550′ acts more passively than the other aforementioned anti-backdrive mechanisms 450, 550 and only when the expansion forces associated with tissue sealing cause the closure pressure between jaw members 510, 520 to fall will anti-backdrive 550′ work to counteract these forces to maintain the closure pressure within the appropriate closure pressure range.

FIG. 7 shows another embodiment of anti-backdrive mechanism 650 for use with forceps 10, 10′. Anti-backdrive mechanism 650 is similar to anti-backdrive mechanisms 450, 550 and, as such, only those differences will be discussed below. Moreover, end effector assembly 600 is similar with respect to FIGS. 6A and 6B and, as such, only those differences are described with respect to the anti-backdrive mechanism 650 are discussed in detail.

Jaw member 610 of end effector assembly 600 cooperates with a drive rod 652 actuatable via handle 40 to move the jaw members 610, 620 between open and closed positions upon translation thereof. Anti-backdrive mechanism 650 includes a pivot pin 603 that is operably supported within jaw member 610. Pivot pin 603 operably connects to a first toggle 615a. A second end of the first toggle 615a connects to pivot pin 605. Pivot pin 605 also connects to a first end of a second toggle 615a. Second toggle 615b pivotably connects at an opposite end to a distal end of the drive rod 652.

In use, actuation of the drive rod 652 in a distal direction initially operates to closed the jaw members 610, 620 under the appropriate sealing force as described above due to the toggles 615a, 615b being in-line with drive rod 652. The handle 40 is configured to move toggle 615b the appropriate amount to provide the requisite closure pressure between jaw members 610, 620 for sealing tissue therebetween.

After the jaw members 610, 620 are closed about tissue, a controller 660 is configured to move the drive rod 652 and toggle 615b to further force toggle 615a distally about pin 603 to increase closure pressure as needed. Based on feedback from controller 660 requesting additional closure pressure between jaw members 610, 620, drive rod 652 forces toggle 615b distally which, in turn, rotates toggle 615a about pivot 603 applying further closure pressure between jaw members 610, 620. The controller 660 may extend the drive rod 652 upon: user request; based on a sealing algorithm; on reaction to feedback from the jaw members 610, 620 or tissue disposed therebetween; or in anticipation of tissue expansion during sealing.

The controller 660 may continually monitor the jaw members 610, 620 for feedback and adjust the drive rod 652 accordingly to maintain the appropriate closure pressure between the jaw members 610, 620 during the entire sealing process. Various types of sensors (not shown) or algorithms may be utilized for this purpose. Once sealed, the drive rod 652 is fully retracted to allow the jaw members 610, 620 to open via handle 40 (or 40′).

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A vessel sealing instrument, comprising:
a housing having a shaft extending from a distal end thereof, a distal end of the shaft having an end effector assembly including a pair of opposing first and second jaw members operably coupled thereto, at least one of the first or second jaw members moveable between an open position and a closed position for clamping tissue with a closure pressure within the range of about 3 kg/cm² to about 16 kg/cm², at least one of the first or second jaw members adapted to connect to a generator configured to provide electrosurgical energy thereto in accordance with a sealing algorithm upon activation thereof; and
an anti-backdrive mechanism operably associated with the end effector assembly, the anti-backdrive mechanism including:
a drive shaft operably coupled at a proximal end to a solenoid and including a distal end configured to operably engage at least one of the first and second jaw members upon extension thereof to provide additional closure pressure between the jaw members, the drive shaft selectively extendible by the solenoid to extend the drive shaft in response to tissue expansion during sealing based on the sealing algorithm.

2. The vessel sealing instrument according to claim 1, wherein, upon extension of the drive shaft in a first direction, the drive shaft engages an abutting surface disposed on a proximal end of the first jaw member.

3. The vessel sealing instrument according to claim 2, wherein the first jaw member is rotatable relative to the second jaw member about a pivot and wherein the abutting surface is offset relative to the pivot.

4. The vessel sealing instrument according to claim 1, wherein, upon extension of the drive shaft in a second direction, the drive shaft retracts relative to the first jaw member allowing the first jaw member to open relative to the second jaw member.

5. The vessel sealing instrument according to claim 1, wherein the solenoid extends the drive shaft in response to tissue expansion during sealing based on the sealing algorithm, the drive shaft configured to maintain the closure pressure between jaw members within the range of about 3 kg/cm² to about 16 kg/cm².
